(19) United States
(12) Patent Application Publication
(10) Pub. No.: US 2023/0172771 A1
(43) Pub. Date: Jun. 8, 2023

(54) GAUZE CUTTING SHEARS

(71) Applicant: Leon Ali Lawson, Flushing, MI (US)

(72) Inventor: Leon Ali Lawson, Flushing, MI (US)

(21) Appl. No.: 17/544,029

(22) Filed: Dec. 7, 2021

(51) Int. Cl. A61F 15/02; B26B 13/28

(52) U.S. Cl. CPC A61F 15/02; B26B 13/28

(57) ABSTRACT

The present invention is an improved design for shears that allows for cutting through gauze or bandages. The inventive shears include first and second levers mounted one to the other by a pivot joint so as to form a fulcrum substantially mid-way along their lengths, and wherein a first end of one of the levers are formed as a shearing blade and wherein the second ends of both of the levers are formed as looped handles mounted on the ends of corresponding stems. The first lever has a cutting edge along one side of its length, with a facing edge of the second lever being blunt, the levers allowing for shearing engagement of the cutting edge over the blunt edge as the levers are rotated relative to one another about the pivot joint between an open position wherein the cutting edge and blunt edge form a V-shaped crease for receiving in the crease an object to be cut, and a closed position wherein the cutting edge substantially overlaps the blunt edge. The second lever has no sharpened edges or points and has an inclined profile from its tip to the pivot joint allowing the second lever to more effectively lift away any material to be cut.

### BACKGROUND OF THE INVENTION

### Field of the Invention

The present invention relates to shears or scissors, More particularly, it is directed to medical shears or scissors that can cut though gauze and medical bandages which are still on the patient.

### Description of the Prior Art

Shears or scissors are useful pieces of equipment for medical personnel including but not limited to emergency medical response, rescue and veterinary professionals. In many instances, the scissors utilized by such medical personnel have been developed for specific applications such as emergency medical technician (“EMT”) shears, bandage, and veterinary scissors. EMT shears, for example, are designed to cut through heavy fabrics such as denim or seatbelt material, which may need to be removed to effect rescue.

Some scissors are designed for extremely specific uses and thus depart from the “normal” or standard scissors in one or more ways. For example, veterinary scissors, one type of specialized scissors, having one smooth blade and one serrated blade, are designed for cutting through cartilage and soft bone. Other shears are designed to cut through heavy fabrics as mentioned above while yet others are designed to cut through meat and bone.

Another type of specialized shears are bandage or gauze cutting shears. These shears are designed to cut through bandages while on the patient. The bandage or gauze is typically relatively thick so a robust construction is required. Also, the design must have some means of limiting injury when in use, as scissors are actually pivotally connected knives which have the ability to do great damage if improperly designed. Typically, these shears have an upper and a lower blade, where the lower blade is fashioned to reduce injury by limiting sharp edges.

While the prior art attempts to reduce injury by having a safety blade have been effective, there is a drawback. The safety blade can be ineffective when the bandage material is close to or actually stuck to the skin of the patient. If the blade, which typically has a blunt tip or leading edge, cannot get under or lift away the bandage it cannot cut the material. Repeated attempts to lift the bandage material, essentially using a “stabbing” motion can result in injury to the patient as any twisting or mishandling of the shears during this motion can cause the user to inadvertently expose the patient to a sharp edge.

Accordingly, it would be desirable to have a bandage or gauze cutting scissors design that overcomes the drawbacks of the prior art.

The present invention is an improved design for shears that allows for cutting through gauze or bandages. The inventive shears include first and second levers mounted one to the other by a pivot joint so as to form a fulcrum substantially mid-way along their lengths, and wherein a first end of one of the levers are formed as a shearing blade and wherein the second ends of both of the levers are formed as looped handles mounted on the ends of corresponding stems. The first lever has a cutting edge along one side of its length, with a facing edge of the second lever being blunt, the levers allowing for shearing engagement of the cutting edge over the blunt edge as the levers are rotated relative to one another about the pivot joint between an open position wherein the cutting edge and blunt edge form a V-shaped crease for receiving in the crease an object to be cut, and a closed position wherein the cutting edge substantially overlaps the blunt edge. The second lever has no sharpened edges or points and has an inclined profile from its tip to the pivot joint allowing the second lever to more effectively lift away any material to be cut.

### Summary of the Invention

It is a major object of the invention to provide an improved design for gauze cutting shears.

It is another object of the invention to provide gauze cutting shears that reduce injury to patients.

It is another object of the invention to provide gauze cutting shears that can help lift and separate bandaging from the patient.

It is another object of the invention to provide gauze cutting shears that have differential levers.

It is another object of the invention to provide gauze cutting shears that have a sharpened lever and a blunt lever.

It is another object of the invention to provide gauze cutting shears that have a blunt lever with an inclined plane.

These and other objects of the present invention will become readily apparent upon further review of the following specification and drawings.

The present invention meets or exceeds all the above objects and goals. Upon further study of the specification and appended claims, further objects and advantages of this invention will become apparent to those skilled in the art.

### BRIEF DESCRIPTION OF THE DRAWINGS

Various other objects, features, and attendant advantages of the present invention will become more fully appreciated as the same becomes better understood when considered with the accompanying drawings, in which like reference characters designate the same or similar parts throughout the several views, and wherein:

FIG. 1 is a plan view of the shears of the invention.

FIG. 2 is a side view of the shears of the invention.

FIG. 3 is a end view of one lever of the shears of the invention.

FIG. 4 is a side view of one lever of the shears of the invention.

### DETAILED DESCRIPTION OF THE PREFERRED EMBODIMENTS

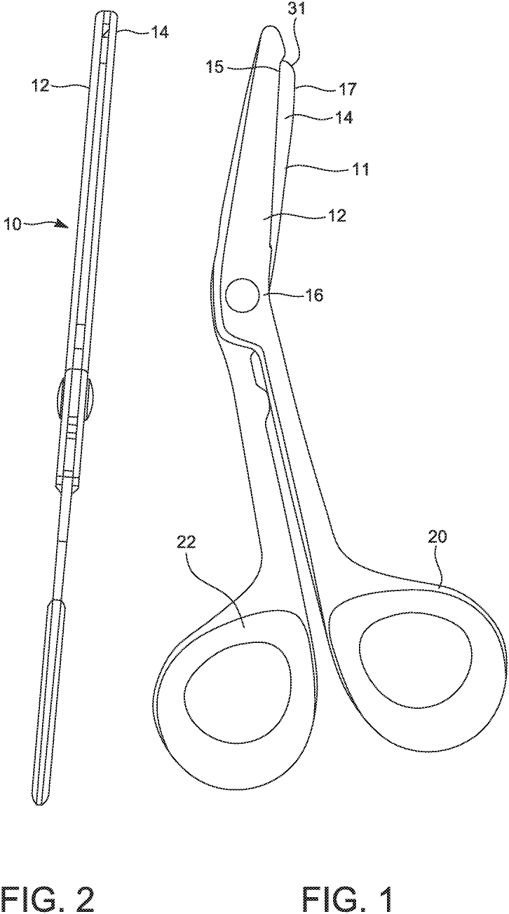

Referring now to FIGS. 1 and 2 it can be seen the inventive shears 10 include substantially linear elongate first and second levers 12 and 14 respectively mounted one to the other by a pivot joint 16 so as to form a fulcrum or pivot substantially mid-way along their lengths.

A first end 15 and 17 respectively of each of said first and second levers 12 and 14 are formed as shearing or cutting blades having cutting edges 9, 11 along opposed facing edges of the first and second levers 12, 14 respectively. The cutting edges are for shearing engagement of one of the cutting edges over the other as the levers are rotated towards each other in directions relative to one another about the fulcrum, that is about pivot point 16, between an open position wherein the cutting edges form a V-shaped nip for receiving an object to be cut, and a closed position such as seen in FIG. 1 wherein the cutting edges substantially overlap one another so as to close the nip along the length of said blades.

A second end 20 and 22 respectively of each of the first and second levers 12 and 14, opposite the first ends 15 and 17, are formed as handles. The handles include a pair of corresponding loops lying substantially adjacent a plane common with the cutting edges when in the closed position and lying substantially in a plane of rotation of the levers when rotated about the fulcrum.

A key aspect of the invention is that the shearing levers 12, 14 are configured to reduce the incidence of injury when using the shears to remove bandages. As is known in the art, shearing blades for removing bandages are specially designed to cut through heavy bandages and cloth while minimizing the potential for injury to the patient. The problem of reducing the potential for injury is exacerbated by the fact the shears 10 must be partially inserted under the material and positioned near the patients skin. While various designs limiting the use of sharp edges are known, no prior art device both limits injury while providing a lift effect when used to cut bandages.

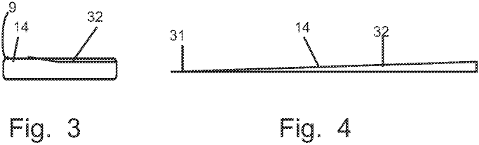

Referring now to FIGS. 3 and 4 a cross section and side view of lever 14 is shown. This lever 14 is the lowermost or bottom lever when operating the shears. This lever 14 has no blade or knife portion and all edges 30 are rounded except non-sharpened edge 11 which cooperates with edge 9 of lever 12 to create the cutting action as explained above. The tip 31 of the 14 is rounded as this is the leading edge of the shears which is not visible when placed beneath, e.g., a bandage. It can be appreciated that if the tip were pointed a user can easily injure someone when pushing the shears under a bandage. Thus lever 14 can be placed against the skin of the patient with no sharp edges touching the patient.

The cutting action for the removal of bandages requires the material to be lifted away from the user. To that end, lever 14 includes an inclined surface 32. It can be seen that this surface 32 is inclined from end 15 to fulcrum 16. This inclined surface 32 helps to lift away bandage material from the skin of the patient, which is a problem often exacerbated by bodily fluids holding the material to the user. In order to create the inclined surface 32 the tip 31 of lever 14 should be made thin relative to the lever portion 33 at the pivot point.

The top lever 12 has a tip 38 which is rounded and has no cutting edge. When in the fully closed position, the cutting edge 9 of lever 12 is positioned in an offset relation to edge 11 of lever 14 so that when fully closed the cutting edge 9 is recessed into a noncontacting position, that is, the edge 9 cannot accidentally injure as it cannot be contacted when the shears are closed. When stored, the shears 10 can be grasped from any angle without risk of injury as the sharp edge 9 is essentially retracted.

In use, the user positions the shears 10 under the material to be cut. Lever 14 is slid under the material and cutting action is initiated by rotating levers 12, 14 using handles 20, 22 as described above. Lever 14 is continually advanced in the length direction, with surface 32 lifting away bandage material as the lever 14 is advanced.

From the foregoing description, one skilled in the art can easily ascertain the essential characteristics of this invention and, without departing from the spirit and scope thereof, can make various changes and modifications of the invention to adapt it to various usages and conditions.

It is to be understood that the present invention is not limited to the sole embodiment described above, but encompasses any and all embodiments within the scope of the following claims:

I claim:

1. A pair of shears comprising:
substantially linear elongate first and second levers attached by a pivot joint so as to form a fulcrum substantially mid-way along their lengths, a first end of each of said first and second levers formed as shearing blades each having cutting edges along opposed facing edges of said first and second levers respectively for shearing engagement of one of said cutting edges over the other as said levers are rotated relative to one another about said fulcrum between an open position wherein said cutting edges form a V-shaped nip for receiving in said nip an object to be cut, and a closed position wherein said cutting edges substantially overlap one another so as to close said nip along the length of said blades;
said second lever having an inclined plane formed thereon.

2. The shears of claim 1 wherein said first lever is an upper lever and cutting edge is sharpened.

3. The shears of claim 1 wherein said second lever is a bottom lever and has no sharpened edges.

4. The shears of claim 2 wherein said upper lever cutting edge is recessed relative to said bottom edge when the shears are in the closed position.

5. The shears of claim 1 wherein said second lever has a rounded tip.
